(19) United States
(12) Patent Application Publication
(10) Pub. No.: US 2023/0000363 A1
(43) Pub. Date: Jan. 5, 2023

(54) CARDIOVASCULAR DETECTION SYSTEM AND METHOD

(71) Applicant: Yi Kai Chen, Taipei (TW)

(72) Inventors: Yi Kai Chen, Taipei (TW); Fu Jen Kao, Taipei (TW); Chen Huan Chen, Taipei (TW); Hao Min Cheng, Taipei (TW)

(21) Appl. No.: 17/856,208

(22) Filed: Jul. 1, 2022

(30) Foreign Application Priority Data
Jul. 2, 2021 (TW) 110124519

(51) Int. Cl. A61B 5/0205; A61B 5/339; A61B 5/346; A61B 5/00

(52) U.S. Cl. CPC A61B 5/0205; A61B 5/339; A61B 5/346; A61B 5/7267; A61B 5/02233

(57) ABSTRACT

A cardiovascular detection system and method, comprising an active compression cuff contracting at a frequency higher than the systolic frequency of the heart. Meanwhile, the detection device is used to capture the influence of the active compression cuff and cardiac systole on the blood of the part to be detected. In addition, it is supplemented by electrocardiography to monitor the reference value of cardiac systole to distinguish the difference between the pulse wave generated by the active compression cuff and the pulse wave generated by the heart. In this way, the state of the cardiovascular system can be quickly understood. Since the active compression cuff is contracted at a frequency higher than the systolic frequency of the heart, it can be more accurately determined whether the blood vessel is blocked or hardened.

### BACKGROUND OF INVENTION

### (1) Field of the Present Disclosure

The present disclosure relates to a cardiovascular detection system and method, and more particularly to a system and a method for detecting heart and blood vessels by use of an active compression cuff that contracts at a frequency higher than the systolic frequency of the heart.

### (2) Brief Description of Related Art

Cardiovascular disease is the most common life-threatening disease after cancer. In detecting cardiovascular diseases, different detection methods are used according to physical conditions, such as blood drawing, electrocardiogram, cardiac ultrasound detection, cardiac computer tomography, etc. The conventional detection methods all require a lot of time for preparation in advance and waiting for the detection result report. Patients undergoing for example: cardiac coronary angiography (CTA) must fast for 6 to 8 hours first, and need to bear the risk of allergy caused by injection of contrast agent. Since the beating frequency of the heart is a fixed bass frequency. At the same time, relying only on the heart sounds emitted by the heart, the detection accuracy is very limited. Furthermore, more detailed and more specific data cannot be detected.

Accordingly, the problem that the detection of cardiovascular diseases takes a lot of time now and how to improve the accuracy of detecting cardiovascular data need to be resolved.

### SUMMARY OF INVENTION

It is a primary object of the present disclosure to provide a cardiovascular detection system and method that ensures a fast, convenient, and high detection accuracy.

According to the present disclosure, an active compression cuff is provided to contract at a frequency higher than the systolic frequency of the heart. Meanwhile, a detection device (such as an electronic stethoscope) is used to capture a physiological information of a part to be detected about the influence of the active compression cuff and the cardiac systole on the blood. In addition, an electrocardiography monitor is employed to monitor an electrocardiogram spectrum information of cardiac contraction, so as to distinguish in the physiological information the pulse wave generated by the active compression cuff and the pulse wave generated by the heart. According to a time difference and a waveform density between waveforms in the physiological information spectrum, it can be determined whether the blood vessel is blocked or hardened. In this way, the detection device of the present disclosure can quickly understand the state of the cardiovascular system of the patient. Moreover, since the active compression cuff is contracted at a frequency higher than the systolic frequency of the heart, it can be more accurately determined whether the blood vessel is blocked or hardened.

### BRIEF DESCRIPTION OF THE DRAWINGS

FIG. 1 is a block diagram I of the structure of a cardiovascular detection system according to the present disclosure;

FIG. 2 is a block diagram II of the structure of a cardiovascular detection system according to the present disclosure;

FIG. 3 is a flow chart of the present disclosure;

FIG. 4 is a schematic diagram I of the implementation of the present disclosure;

FIG. 5 is a schematic diagram II of the implementation of the present disclosure;

FIG. 6 is a schematic diagram III of the implementation of the present disclosure;

FIG. 7 is a schematic diagram IV of the implementation of the present disclosure; and

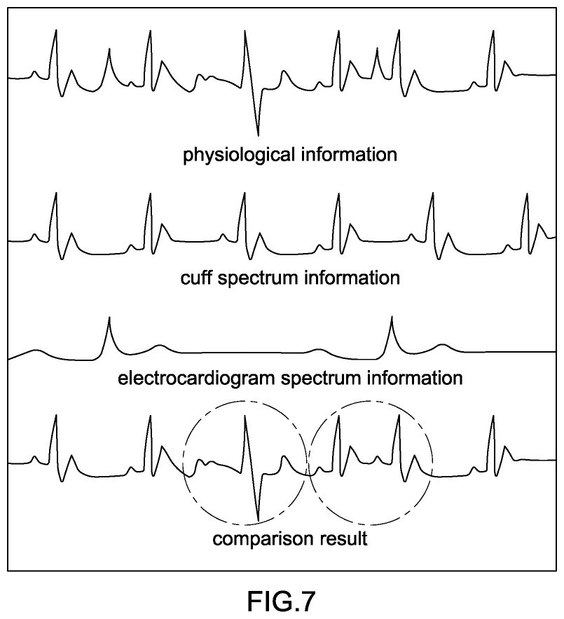

FIG. 8 is a schematic diagram V of the implementation of the present disclosure.

### DETAILED DESCRIPTION OF PREFERRED EMBODIMENTS

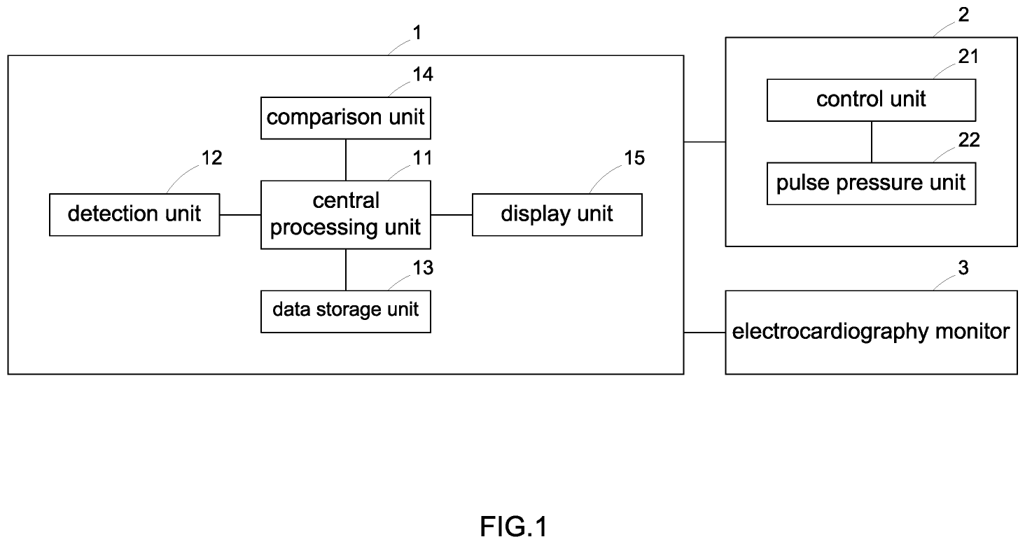

Referring to FIG. 1, a system for detecting heart and blood vessels according to the present disclosure includes a detection device 1, which is in information connection with an active compression cuff 2 and an electrocardiography monitor 3. The detection device 1 can be used to detect the cardiovascular condition of a patient, and mainly includes a central processing unit 11, which is in information connection with a detection unit 12, a data storage unit 13, a comparison unit 14, and a display unit 15. The active compression cuff 2 can be used to continuously compress the patient's blood vessels at a contraction frequency higher than the systolic frequency of the heart for a certain period of time. The active compression cuff 2 includes a control unit 21 which is electrically connected with a pulse pressure unit 22. The electrocardiography monitor 3 can be used to measure the electrophysiological activity of the heart of the patient.

The central processing unit 11 can be used to drive all units of the detection device 1, and has the functions of receiving and transmitting information signals, logical operations, temporary storage of operation results, and storage of execution command positions. It can be a central processing unit (CPU) or a microcontroller unit (MCU).

The detection unit 12 can be one or more vibration sensors. It employs the oscillometric method to acquire the physiological information of the patient. The physiological information may include spectrograms of systolic, diastolic, and mean pressures of blood flowing from the apical artery to the radial artery to cause the vibration of the vessel wall.

The data storage unit 13 can be used to store electronic data, such as a cuff spectrum information, an electrocardiogram spectrum information, a disease symptom information, etc. It can be a solid state disk or solid state drive, a hard disk drive, a static random access memory, a random access memory, a cloud drive, or a combination thereof. The cuff spectrum information includes the cuff spectrogram generated by the active compression cuff 2 corresponding to the contraction frequency. For example, if the active compression cuff 2 is set to contract three times per second, the cuff spectrogram is the one with the contraction frequency of 3 Hz. The electrocardiogram spectrum information includes the electrocardiogram spectrogram obtained by measuring patients (having such as different genders, ages, or various physiological diseases) through the electrocardiography monitor 3. The disease symptom information is the physiological symptoms corresponding to various physiological diseases (such as blockage or hardening of blood vessels, resulting in slow blood flow), the cuff spectrum information and the electrocardiogram spectrum information corresponding to various physiological diseases (for example, if the blood vessel is blocked or hardened, the waveform of the spectrogram will produce time difference or dense waveform), and a detection information obtained by use of the active compression cuff 2 and the electrocardiography monitor.

The comparison unit 14 is used to compare the physiological information (including a time difference and a waveform density between the waveforms in the spectrogram) captured by the detection unit 12 with the electrocardiogram spectrum information (measured synchronously with the electrocardiography monitor 3), the cuff spectrum information corresponding to the contraction frequency, and the disease symptom information, thereby producing a comparison result about the suspected physiological symptoms of the patient. In addition, the electrocardiogram spectrum information is used as a time reference value of cardiac systole to synchronously correct the time axis of the cuff spectrum information corresponding to the contraction frequency.

The display unit 15 can be used to present any received information or its spectrogram, such as the physiological information, the electrocardiogram spectrum information, the cuff spectrum information, and the disease symptom information, etc., so that the user can further analyze the physiological symptoms of the patient.

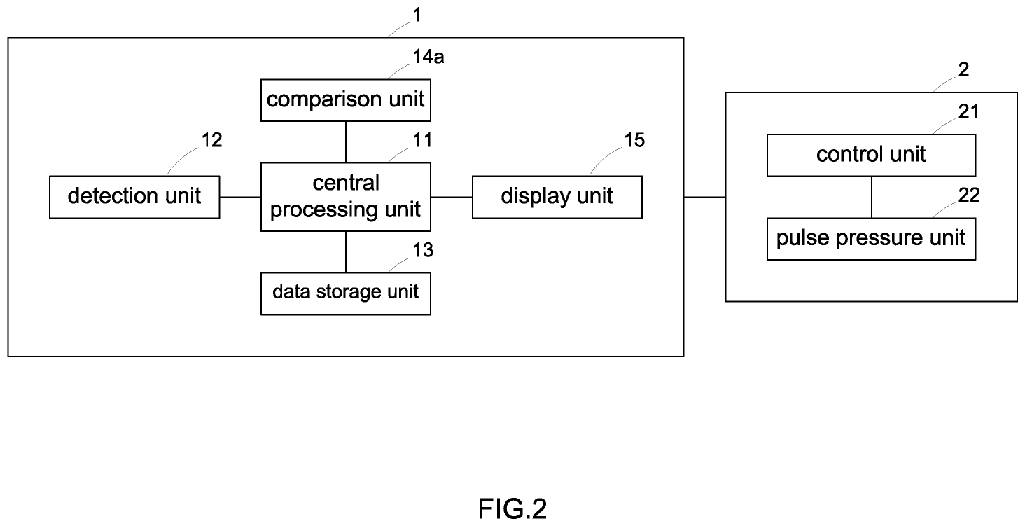

FIG. 2 shows another embodiment of the present disclosure. The difference between another embodiment and the above-mentioned embodiment is that the cardiovascular detection system of the present disclosure mainly includes a detection device 1, which is only in information connection with the active compression cuff 2. The comparison unit 14a of the detection device 1 can be an artificial intelligence unit, which can be trained and learned through machine learning such as supervised learning, semi-supervised learning, reinforcement learning, unsupervised learning, self-supervised learning or heuristic algorithms, but not limited thereto.

The comparison unit 14a uses a plurality of basic information of different persons pre-stored in the data storage unit 13 as input data. Basic information may include gender, age, or physical condition, etc., but not limited thereto. The corresponding electrocardiogram spectrum information is used as the target data for conducting a first machine learning to solve the doubts about the individual differences in cardiovascular function. Next, the comparison unit 14a uses a plurality of cuff spectrum information (corresponding to the contraction frequency of the active compression cuff) pre-stored in the data storage unit 13 as input data. A plurality of disease symptom information about cardiovascular disease can be used as target data for conducting a second machine learning to establish a detection model. The comparison unit 14a removes the electrocardiogram spectrum information from the physiological information captured by the detection unit 12, and generates a retained information about the active compression cuff 2 having influence. Moreover, the detection model compares the retained information and the cuff spectrum information according to the time difference and the waveform density. Then, a difference result obtained by the comparison is compared with the disease symptom information. Finally, a comparison result about the suspected physiological symptoms of the patient is obtained. For example, if there is a time difference between waveforms, it means that the blood cannot return within the normal time, and it is judged that the blood vessel is blocked by blood lipids. If the waveform density is too high, it means that the blood can only pass through the narrowed blood vessels, resulting in higher frequency vibration/fluctuation, which means that the blood vessels have poor elasticity. However, the present disclosure is not limited thereto.

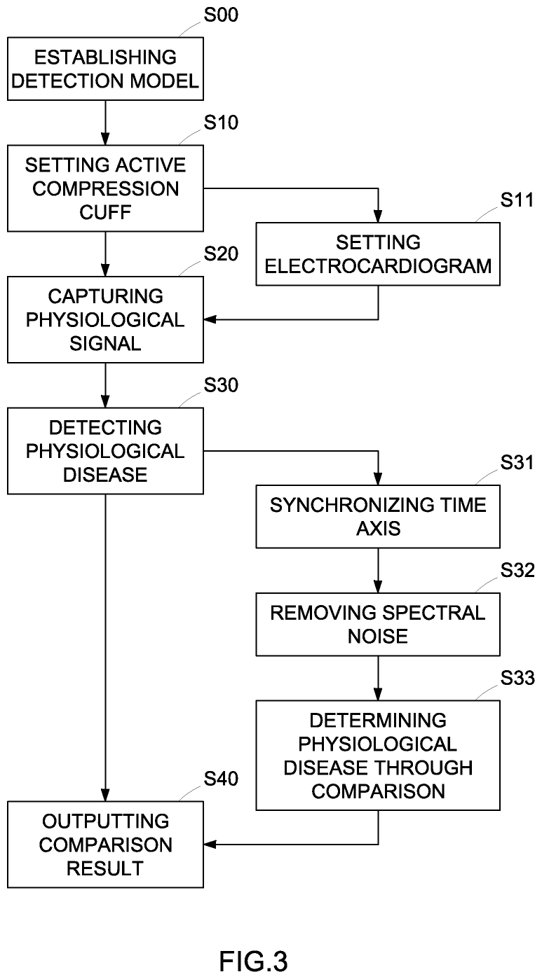

FIG. 3 shows the flowchart of the cardiovascular detection method. The method includes the following steps:

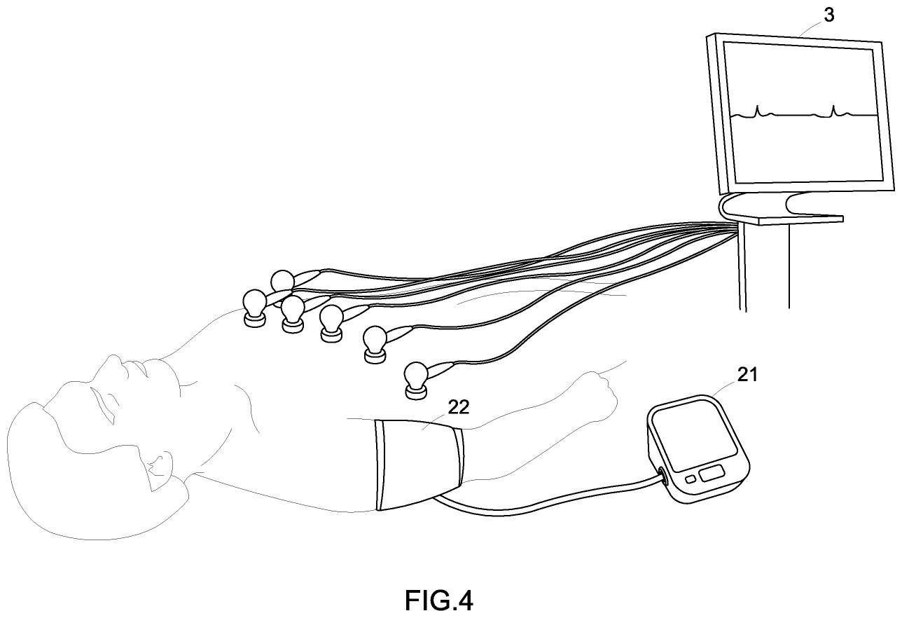

Step S10 of setting an active compression cuff: As shown in FIG. 4, an active compression cuff 2 is fixed on a patient U. A pulse pressure unit 22 is set through a control unit 21 to repeatedly inflate and deflate (pressurize and depressurize) within a certain period of time according to a systolic frequency. The contraction frequency is higher than the systolic frequency of the heart. In performing the step S10 of setting an active compression cuff, another step S11 of setting electrocardiogram is simultaneously performed. The electrocardiography monitor 3 is used to record the electrophysiological activity of the heart of the patient U. The electrocardiography monitor 3 transmits the measured electrocardiogram spectrum information to the detection device 1 synchronously and stores it in a data storage unit 13 of the detection device 1.

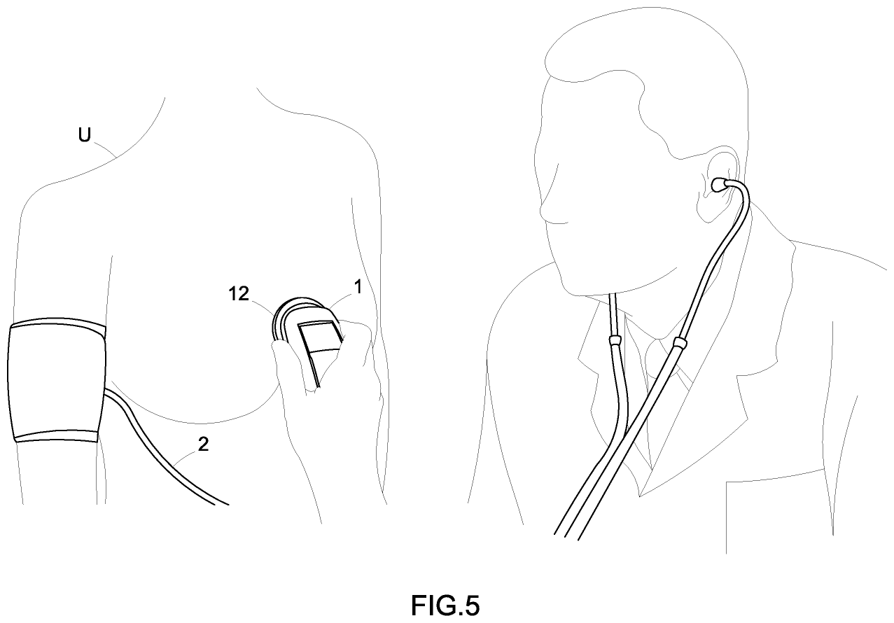

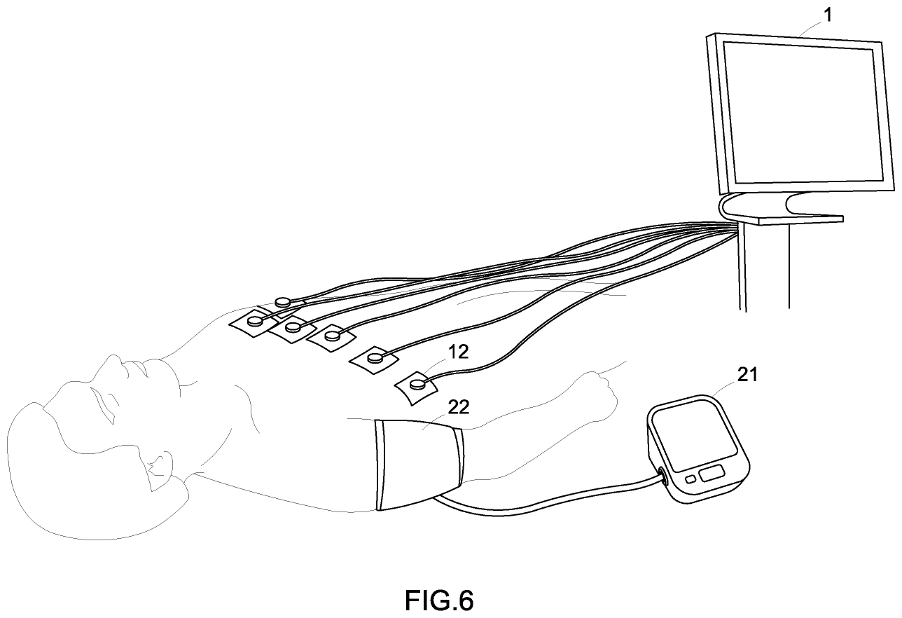

Step S20 of capturing a physiological signal: As shown in FIG. 5, the detection device 1 can be an electronic stethoscope. A detection unit 12 of the detection device 1 is placed on a part to be detected of the patient U. A physiological information obtained by the oscillometric method may include spectrograms of systolic, diastolic, and mean pressures of blood flowing from the apical artery to the radial artery to cause the vibration of the vessel wall. FIG. 6 shows another embodiment of the present disclosure. The detection unit 12 of the detection device 1 can be a plurality of patch-type vibration sensors to capture the physiological information of a plurality of parts to be detected of the patient U. In this way, the electronic stethoscope only using one vibration sensor can be replaced so as to reduce the time for the user to repeat the operation.

The step S30 of detecting a physiological disease: A time difference and a waveform density between waveforms in the physiological information spectrogram are compared through the comparison unit 14 of the detection device 1 with the electrocardiogram spectrum information synchronously measured by the electrocardiograph 3, the cuff spectrum information (corresponding to the contraction frequency) and disease symptom information in the data storage unit 13, thereby obtaining a comparison result about the suspected physiological symptoms of the patient U.

In performing the step S30 of detecting the physiological disease, another step S31 of synchronizing a time axis. The comparison unit 14 uses the electrocardiogram spectrum information as a time reference value to synchronously correct the time axis of the cuff spectrum information corresponding to the contraction frequency, so as to distinguish the pulse wave generated by the active compression cuff 2 from the pulse wave generated by the heart in the physiological information. Meanwhile, another step S32 of removing a spectral noise is performed. The comparison unit 14 removes the electrocardiogram information from the physiological information spectrum, and generates a retained information about the active compression cuff 2 having influence. In addition, a further step S33 of determining the physiological disease through comparison is performed. The comparison unit 14 compares the retained information about the active compression cuff 2 having influence with the cuff spectrum information according to the time difference and the waveform density in the retained information. Then, a difference result obtained by the comparison is compared with the disease symptom information. Finally, a comparison result about the suspected physiological symptoms of the patient U is obtained. For example, if there is a time difference between waveforms, it means that the blood cannot return within the normal time, and it is judged that the blood vessel is blocked by blood lipids. If the waveform density is too high, it means that the blood can only pass through the narrowed blood vessels, resulting in higher frequency vibration/fluctuation, which means that the blood vessels have poor elasticity. Since the contraction frequency of the active compression cuff is greater than the systolic frequency of the heart, the user can more accurately determine whether the blood vessel is blocked or hardened according to the waveform in the comparison result.

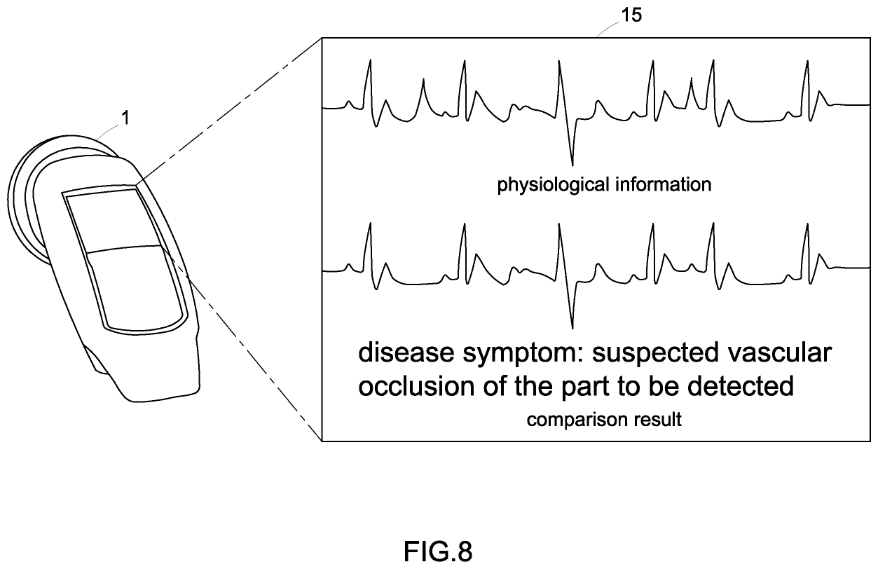

Step S40 of outputting a comparison result: Referring to FIG. 8, the physiological information and the comparison result are presented through a display unit 15 of the detection device 1, so that the user can know and observe the physiological condition of the patient U, and record it as the disease symptom information in the data storage unit 13.

In another embodiment, before performing the step S10 of setting the active compression cuff, a further step S00 of establishing a detection model is performed in advance: A comparison unit 14 of the detection device 1 conducts training and learning through machine learning. A plurality of pieces of basic information (stored in the data storage unit 13) about different persons are used as input data. The basic information can be gender, age, or physical condition, etc., but not limited thereto. The corresponding electrocardiogram spectrum information is used as the target data for conducting a first machine learning to solve the doubts about the individual differences in cardiovascular function. Next, a plurality of cuff spectrum information (corresponding to the contraction frequency of the active compression cuff) pre-stored in the data storage unit 13 is used as input data. A plurality of disease symptom information about cardiovascular disease can be used as target data for the comparison unit 14 to conduct a second machine learning, thereby establishing a detection model.

In addition, the step S30 of detecting the physiological disease is performed through the detection model. The comparison unit 14 removes the electrocardiogram spectrum information from the physiological information captured by the detection unit 12, and generates a retained information about the active compression cuff 2 having influence. Moreover, the detection model compares the retained information and the cuff spectrum information according to the time difference and the waveform density. Then, a difference result obtained by the comparison is compared with the disease symptom information. Finally, a comparison result about the suspected physiological symptoms of the patient is obtained. For example, if there is a time difference between waveforms, it means that the blood cannot return within the normal time, and it is judged that the blood vessel is blocked by blood lipids. If the waveform density is too high, it means that the blood can only pass through the narrowed blood vessels, resulting in higher frequency vibration/fluctuation, which means that the blood vessels have poor elasticity.

According the present disclosure, the active compression cuff is contracted at a frequency higher than the systolic frequency of the heart. Meanwhile, the detection device is used to capture the physiological information of the part to be detected of the patient. After removing the electrocardiogram spectrum information from the physiological information, the difference between the physiological information and the normal spectrum information that should be produced by the active compression cuff is determined through comparison according to the time difference and waveform density between the waveforms. Then, the difference is compared with the disease symptom information to identify whether the blood vessel is blocked or hardened. In this way, the state of the cardiovascular system of the patient can be quickly obtained through the detection device of the present disclosure. Meanwhile, a large amount of time cost can be reduced. Since the active compression cuff is contracted at a frequency higher than the systolic frequency of the heart, the accuracy of detecting cardiovascular-related data can be improved. As a result, a cardiovascular detection system and method can achieve a fast, convenient, and high detection accuracy.

### REFERENCE SIGN

1 detection device
11 central processing unit
12 detection unit
13 data storage unit
14, 14a comparison unit
15 display unit
2 active compression cuff
21 control unit
22 pulse pressure unit
3 electrocardiography monitor
U patient
S00 establishing detection model
S10 setting active compression cuff
S11 setting electrocardiogram
S20 capturing physiological signal
S30 detecting physiological disease
S31 synchronizing time axis
S32 removing spectral noise
S33 determining physiological disease through comparison
S40 outputting comparison result

What is claimed is:

1. A cardiovascular detection system, comprising a detection device in information connection with an active compression cuff, the active compression cuff being used to contract according to a contraction frequency, the detection device having a central processing unit in information connection with a detection unit, a data storage unit, a comparison unit, and a display unit,
wherein the detecting unit is used for obtaining a physiological information of a patient;
wherein the comparison unit is used for comparing the physiological information with a disease symptom information in the data storage unit and a cuff spectrum information corresponding to the contraction frequency according to a time difference and a waveform density between waveforms of the physiological information, thereby creating a comparison result; and
wherein the display unit is used for displaying the physiological information and the comparison result.

2. The cardiovascular detection system as claimed in claim 1, wherein the detection device is simultaneously connected with an electrocardiography monitor, and wherein the electrocardiography monitor is used to acquire an electrocardiogram spectrum information of the patient, and wherein the electrocardiogram spectrum information is used as a time reference value to synchronously correct a time axis of the cuff spectrum information.

3. The cardiovascular detection system as claimed in claim 2,
wherein the comparison unit removes the electrocardiogram spectrum information from the physiological information to generate a retained information;
wherein the retained information and the cuff spectrum information are compared according to the time difference and the waveform density of the retained information, thereby generating a difference result; and
wherein the difference result is compared with the disease symptom information to generate the comparison result.

4. The cardiovascular detection system as claimed in claim 1,
wherein the comparison unit is an artificial intelligence unit; and
wherein the comparison unit performs a first machine learning through a plurality of pieces of basic information and a plurality of pieces of electrocardiogram spectrum information corresponding to the basic data and stored in the data storage unit.

5. The cardiovascular detection system as claimed in claim 4,
wherein the comparison unit removes the electrocardiogram spectrum information from the physiological information and generates a retained information.

6. The cardiovascular detection system as claimed in claim 5,
wherein the comparison unit uses a plurality of pieces of cuff spectrum information (corresponding to the contraction frequency) pre-stored in the data storage unit and a plurality of pieces of disease symptom information to conduct a second machine learning, thereby establishing a detection model;
wherein the detection model is used to compare the retained information with the cuff spectrum information according to the time difference and the waveform density, thereby creating a difference result; and
wherein the detection model is used to compare the difference result with the disease symptom information to generate the comparison result.

7. The cardiovascular detection system as claimed in claim 1, wherein the contraction frequency is higher than the systolic frequency of the heart.

8. The cardiovascular detection system as claimed in claim 1, wherein the detection unit is a plurality of patch-type vibration sensors.

9. A cardiovascular detection method, comprising the following steps:
fixing an active compression cuff on a patient and setting it to contract according to a contraction frequency;
placing a detection unit of a detection device on a part to be detected of the patient for capturing a physiological information;
comparing the physiological information by use of a comparison unit with a disease symptom information in a data storage unit and a cuff spectrum information corresponding to the contraction frequency according to a time difference and a waveform density between waveforms of the physiological information, thereby creating a comparison result; and
displaying the physiological information and the comparison result through the display unit.

10. The cardiovascular detection method as claimed in claim 9, further comprising:
fixing electrode patches of an electrocardiography monitor onto the patient to capture an electrocardiogram spectrum information and transmit it to the detection device; and
correcting a time axis of the cuff spectrum information synchronously through the comparison unit by use of the electrocardiogram spectrum information as a time reference value.

11. The cardiovascular detection method as claimed in claim 10, further comprising:
removing the electrocardiogram spectrum information by the comparison unit from the physiological information to generate a retained information;
comparing by the comparison unit the retained information with the cuff spectrum information according to the time difference and the waveform density, thereby creating a difference result; and
compare by the comparison unit the difference result with the disease symptom information to generate the comparison result.

12. The cardiovascular detection method as claimed in claim 9,
wherein the comparison unit is an artificial intelligence unit; and
wherein the comparison unit performs a first machine learning through a plurality of pieces of basic information and a plurality of pieces of electrocardiogram spectrum information corresponding to the basic data and stored in the data storage unit.

13. The cardiovascular detection method as claimed in claim 12, wherein the comparison unit removes the electrocardiogram spectrum information from the physiological information to generate a retained information.

14. The cardiovascular detection method as claimed in claim 13,
wherein the comparison unit uses a plurality of pieces of cuff spectrum information (corresponding to the contraction frequency) pre-stored in the data storage unit and a plurality of pieces of disease symptom information to conduct a second machine learning, thereby establishing a detection model;
wherein the detection model is used to compare the retained information with the cuff spectrum information according to the time difference and the waveform density, thereby creating a difference result; and
wherein the detection model is used to compare the difference result with the disease symptom information to generate the comparison result.

15. The cardiovascular detection method as claimed in claim 9, wherein the contraction frequency is higher than the systolic frequency of the heart.
